(19) United States
(12) Patent Application Publication
(10) Pub. No.: US 2024/0417664 A1
(43) Pub. Date: Dec. 19, 2024

(54) METHOD FOR CLEANING A MICROFLUIDIC DEVICE USING AN IONIC LIQUID

(71) Applicant: LUMICKS CA HOLDING B.V., Amsterdam (NL)

(72) Inventor: Rui Pedro RIJO DA COSTA CARVALHO, Amsterdam (NL)

(73) Assignee: LUMICKS CA HOLDING B.V., Amsterdam (NL)

(21) Appl. No.: 18/721,223

(22) PCT Filed: Dec. 20, 2022
(86) PCT No.: PCT/EP2022/087004

§ 371 Date: Jun. 18, 2024

(30) Foreign Application Priority Data
Dec. 21, 2021 (NL) 2030210

(51) Int. Cl. C12M 1/00; B08B 9/00; C11D 7/50; C12M 3/06

(52) U.S. Cl. CPC C12M 39/00; B08B 9/00; C11D 7/5013; C12M 23/16; B08B 2209/00

(57) ABSTRACT

The invention pertains to a method for cleaning a microfluidic device. More specifically the invention pertains to a method for cleaning a microfluidic device, wherein the method comprises removing biological matter from the inside of the device by flushing the microfluidic device with an ionic liquid. The invention further pertains to the use of an ionic liquid to clean mammalian cells and to a kit of parts comprising a microfluidic device and an ionic liquid.

### FIELD OF THE INVENTION

The invention pertains to a method for cleaning a microfluidic device. More specifically the invention pertains to a method for cleaning a microfluidic device, wherein the method comprises removing biological matter from the inside of the device by flushing the microfluidic device with a cleaning agent and wherein the cleaning agent comprises an ionic liquid.

### BACKGROUND OF THE INVENTION

The study of cell interactions, such as the binding strength between two cells or between cells and biomolecules, is a highly relevant and active research area in the biosciences. An important parameter is the avidity, which characterizes the cumulative effect of multiple individual binding interactions between cells. Another important parameter is the affinity, which characterizes the strength with which one molecule binds to another molecule, for example the strength with which a receptor on the cell membrane of an immune cell binds to an antigen on the target cell. The avidity and affinity play an essential role in the study and development of therapies in medicine, such as immune oncology and immunology in general.

A known technique for studying cell adhesion to biomolecules and for studying interaction strengths between cells is referred to as acoustic force spectroscopy (AFS). This technique allows to study interactions between cells and a functionalised surface by applying an acoustic force to the cells. Kamsma et al. reported in their article ‘Single-cell acoustic force spectroscopy: resolving kinetics and strength of T cell adhesion to fibronectin’, 2018, Cell Reports 24, 3008-3016, 11 Sep. 2018 on a study related to the adhesion of T cells to fibronectin using an acoustic force spectroscopy AFS system.

Similarly, WO2018/083193 describes an AFS system including a microfluidic device comprising a so-called functionalised wall surface which may include target cells. A plurality of unlabelled effector cells, e.g. T-cells, can be flushed into the microfluidic device, so that they can settle and bind to target cells. Thereafter, an acoustic source is used to exert a ramping force on the bound effector cells so that effector cells, at a certain force, will detach from the target cells. During this process, the spatiotemporal behaviour of the effector cells in the microfluidic device is imaged using an imaging microscope. The interaction between cells, e.g. the force at which the effector cells detach, may be determined by analysing the captured video images. For example, the cell avidity between the effector cells and the target cells can be determined this way.

After having performed a (series of) measurement(s) in an AFS system, the microfluidic device needs to be cleaned before starting a subsequent measurement. Or, more specifically, all biological matter deposited on the inside of the device, which includes both the target cells and the effector cells, needs to be removed before stating a subsequent measurement. In a typical case, the inside of the device is at least partly covered by adsorbed target cells. Adsorbed onto the target cells there might be effector cells. In this case, the cleaning of the microfluidic device encompasses removal of the effector cells and the target cells as well all biological matter that is deposited by the cells.

It is thus clear that it is beneficial to have a method for cleaning the inside of a microfluidic device such that it can be re-used for a subsequent experiment. The device typically comprises a surface of particular relevance on the inside. This may be the surface on which a measurement is performed, but it may also be a contact surface in a sorting device. It is key that the inside of the device, and in particular the surfaces of particular relevance, is cleaned in such a manner that it can be re-used in a subsequent experiment. This means that the inside of the device, and in particular the relevant surfaces inside the device, is in a state equal to the state before the first use. Ideally, this is irrespective of the number of cleaning cycles. Hence, all cellular material needs to be removed, but no further material, such as material from the inner surfaces, may be removed. Also, the surface properties inside the device may not be changed.

In case of a microfluidic device that is used in an AFS system, it is typical that the device after use comprises layers of effector and target cells. This means that cleaning implies that the device is cleaned on the inside by removing all cellular material such that the inside surfaces are again in a state equal to that before the first use (i.e. before the first application of the cells), whilst maintaining the dimensions and surface properties of the cell.

Methods of cleaning a microfluidic device are known. It may be possible to disassemble the microfluidic device and clean it using any known method, which includes physical scrubbing. Alternatively, there are known methods to chemically clean the device in-situ, i.e. without disassembling or opening it. It is, for instance, described in US2013186433A1 that acid solutions, solutions containing oxidizers or bleach can be applied to clean nucleic acid from a microfluidic device. US2005019213A1 describes how to clean a microfluidic cartridge using NaOH. The current methods for cleaning a microfluidic device hence require disassembly or the use of caustic and/or corrosive chemicals such as strong acids/bases or bleach. Disassembly is cumbersome and prone to all sorts of wear and failures of the device. Also, some microfluidic devices are formed from a single piece of material and cannot be disassembled without destruction of the device. Current chemical cleaning methods are dangerous due to the corrosive and/or volatile nature of the chemicals and are therefore preferably avoided.

Also, and perhaps most importantly, the caustic and/or corrosive chemicals can etch the inner surfaces of the device which can influence its geometry as well as negatively affect other important device properties (e.g. hydrodynamic, optical, electrical or acoustic properties). In addition, if the device contains metal electrodes on the inside, the chemical may cause corrosion of said electrodes leading to electrical malfunction. In acoustic devices, an optimal force generation may be achieved by selecting acoustic cavity parameters and wavelength of the acoustic wave in order to create a maximum pressure gradient at the functionalised wall surface by ensuring that the distance from the wall surface to the acoustic node is approximately % wavelength. This means that if the acoustic resonance properties of the device change, there will be a reduction in its performance. As a result, the device can only be cleaned a limited number of times before the acoustic properties are altered to such an extent that it can no longer be used for performing reliable measurements.

Hence, there is a need for a better, faster and easier method of cleaning the inside of a microfluidic device without the use of caustic or corrosive chemicals. This method is preferably in-situ, meaning that there is no need to disassemble the microfluidic device. It is further preferred that it does not require the use of elaborate safety measures, such as ventilated fume hoods. Additionally, it is also preferred that the cleaning method does not damage the microfluidic device or the associated fluidic and/or electrical connections. More specifically, it is preferred that the dimensions and/or the acoustic properties of the microfluidic device are not affected by the cleaning method or cleaning agents such that it can be cleaned and re-used for a large number of times. These and other problems are solved by the current invention.

### SUMMARY OF THE INVENTION

The invention relates to a method for cleaning a microfluidic device, wherein the method comprises removing biological matter, such as mammalian cells, mammalian cell organelles, mammalian cell residues and/or extracellular matrix from mammalian cells, from the inside of the microfluidic device by flushing the microfluidic device with a cleaning agent that comprises an ionic liquid. Ionic liquids are salts that are in the liquid state and find applications in many technological areas such as catalysis and electrical applications such as novel batteries. When using an ionic liquid to clean a microfluidic device, the ionic liquid can be flushed though the device without any need for disassembling or opening the device. In other words, the ionic liquid can be used to clean the device in-situ. In-situ cleaning has many advantages, including that it is typically faster than cleaning methods that do require disassembling a device. Also, disassembling a device often causes wear and tear on the disassembled parts compromising their expected operational life.

Ionic liquids are generally considered to be non-corrosive and non-toxic. Also, they have a low vapour pressure meaning that they are non-volatile and/or hardly evaporate. As a result, ionic liquids are a safer and more environmentally friendly alternative for the corrosive cleaning liquids that are known from the prior art.

It has been found that ionic liquids do not damage nor etch the microfluidic device. Corrosive chemicals, on the other hand, are known to etch away the surfaces inside the microfluidic device causing a change in the acoustic, hydrodynamic, optical, and electrical properties. Typically, when using corrosive chemicals, a microfluidic device can only be cleaned about ten times. More cleaning cycles are not advised as the acoustic properties are changed to such an extent that the measurement becomes inaccurate. As a result, the microfluidic device needs to be replaced with a new device which is wasteful, expensive, and not sustainable.

It has been found that ionic liquids do not damage the microfluidic device, nor do they damage the associated tubing, connectors, and other parts. Using ionic liquids, the device can be cleaned without affecting the acoustic, hydrodynamic, optical, and/or electrical properties, meaning that the device can be cleaned many times before there is a need for a replacement.

The invention further relates to the use of a cleaning agent to remove mammalian cells, mammalian cell residues and/or extracellular matrix from a surface, wherein the cleaning agent comprises an ionic liquid.

Also, the invention relates to a kit of parts comprising a microfluidic device and a container holding an ionic liquid. Preferably, the kit further comprises an aqueous soap solution.

### Embodiments According to the Invention

1. A method for cleaning a microfluidic device, wherein the method comprises removing biological matter from the inside of the microfluidic device by flushing the microfluidic device with a cleaning agent, characterized in that the cleaning agent comprises an ionic liquid.
2. The method according to embodiment 1, wherein the biological material comprises mammalian cells, mammalian cell organelles, mammalian cell residues and/or extracellular matrix from mammalian cells.
3. The method according to embodiment 1, wherein the biological material consists of mammalian cells and/or extracellular matrix from mammalian cells 4. The method according to embodiment 2 or 3, wherein the mammalian cells are target cells and/or effector cells.
5. The method according to any of the previous embodiments, wherein the cleaning method is an in-situ cleaning method.
6. The method according to embodiment 2-5, wherein the mammalian cells are lysed by the cleaning agent.
7. The method according to any one of the previous embodiments, wherein the ionic liquid comprises at least one cation selected from 1-butyl-1-methyl-pyrrolidinium, 1-ethyl-3-methylimidazolium, and methyltrioctylammonium.
8. The method according to any one of the previous embodiments, wherein the ionic liquid comprises at least one anion selected from chloride, bromide, iodide, acetate, nitrate, sulfate, sulfonate, tosylate, dicyanamide, thiocyanate, borate, and salicylate.
9. The method according to any one of the previous embodiments, wherein the contact time is at most 960 minutes, preferably at most 60 minutes, and even more preferably at most 30 minutes.
10. The method according to any one of the previous embodiments, wherein the contact time is at least 5 minutes, preferably at least 10 minutes, and even more preferably at least 20 minutes.
11. The method according to any one of the previous embodiments, wherein the temperature of the ionic liquid is between 15 and 100° C., preferably between 3° and 50° C.
12. The method according to any one of the previous embodiments, further comprising flushing the microfluidic device with an aqueous soap solution.
13. The method according to any one of the previous embodiments, further comprising flushing the microfluidic device with water.
14. The method according to any one of the previous embodiments, wherein the cleaned device is reused.
15. The method according to any one of the previous embodiments, wherein the device can be cleaned at least 10 times, more preferably at least 50 times, even more preferably at least 100 times, and even more preferably at least 150 times without altering the acoustic, hydrodynamic, optical, and/or electrical properties of the device.
16. Use of a cleaning agent to remove mammalian cells, mammalian cell residues and/or extracellular matrix from a surface, wherein the cleaning agent comprises an ionic liquid.
17. The use according to embodiment 16, wherein the cleaning agent consist of ionic liquid.
18. The use according to any one of embodiments 16-17, wherein the ionic liquid comprises at least one cation selected from 1-butyl-1-methyl-pyrrolidinium, 1-ethyl-3-methylimidazolium, and methyltrioctylammonium.
19. The use according to any one of embodiments 16-18, wherein the ionic liquid comprises at least one anion selected from chloride, bromide, iodide, acetate, nitrate, sulfate, sulfonate, tosylate, dicyanamide, thiocyanate, borate, and salicylate.
20. A kit of parts comprising a microfluidic device and a container holding an ionic liquid.
21. The kit of parts according to embodiment 20, wherein the kit of parts further comprises a container holding an aqueous soap solution.

### DETAILED DESCRIPTION

The current invention relates to a method for cleaning a microfluidic device, wherein the method comprises removing biological matter from the inside of the microfluidic device by flushing the microfluidic device with a cleaning agent, wherein the cleaning agent comprises an ionic liquid. Preferably, the cleaning agent consists of ionic liquid, more preferably a single ionic liquid.

It is understood that a microfluidic device is a device that may contain a liquid and that flushing the device involves a liquid that flows through the device. It is understood that in this context, cleaning refers to removing biological matter such as cellular layers or cellular material, which are understood not to be part of the device, from the inside of the device without damaging the device or its inside surfaces. This means that the priming layer, which can be a physiosorbed coating (e.g. fibronectin, PLL) or a covalent coating (e.g. silane) may remain inside the device. In other words, cleaning refers to restoring the device to a base state where it does not comprise any cellular layer(s) or deposited cellular material. Here a base state can be interpreted as the state before the first use of the device.

In a preferred embodiment, the cleaning method is an in-situ cleaning method. In the context of the current invention, the term in-situ cleaning of a microfluidic device refers to the cleaning of said microfluidic device without disassembling or opening it. It is understood that in-situ cleaning does include the case where the entire microfluidic device is taken out of the larger device in which it is mounted for performing its function (i.e. measuring cell properties, sorting cells, etc.) and inserted into a cleaning apparatus without disassembling or opening the microfluidic device itself.

It is further understood that flushing, in the context of the current invention, refers to allowing a liquid, such as the cleaning agent or the ionic liquid, to flow through the cell for a certain period of time. The force or energy needed for the liquid to flow can be imposed by any known method, such as pumping, sucking, gravity, and capillary force. The minimum time for performing a flush is that time needed to replace all liquid inside the cell by the liquid with which the cell is flushed. However, typically many times the volume of the cell is flushed through the cell.

In a preferred embodiment, the liquid used to flush the device is at least partly recycled. This can be achieved by rerouting at least part of the fluid from the outlet of the device to its inlet and thereby creating a loop. Alternatively, it is possible to allow the liquid to flow forward and backward, i.e. by amending the direction of the flow. It is further understood that flushing the microfluidic device may imply stopping the flow of liquid for a certain time to allow for slower phenomena to occur, such as possibly the lysing of cells or the detachment of biological material from the surfaces inside the device.

It is further understood that the flow of the cleaning agent can exert a shear force on the adsorbed material which may aid in the cleaning action of the method. It is hence preferred that the cleaning agent is flowing for at least 50% of the time of the flush, preferably at least 60% of the time of the flush, preferably least 70% of the time of the flush and more preferably least 80% of the time of the flush. Also, it is preferred that the shear force exerted by the cleaning agent onto the adsorbed material is at least 0.1 Pa, preferably at least 0.3 Pa, more preferably at least 0.5, and even more preferably at least 1 Pa.

In the context of the current invention, a microfluidic device is a microfluidic device for laboratory testing. Depending on the context, these devices are also denoted as lab-on-a-chip, flow cells or fluidic chips. The device has at least one inlet and one outlet in such a manner that a liquid can enter the device from the inlet, flow through the device and exit the device from the outlet. Typically, the device has a certain degree of symmetry and the terms inlet and outlet are only defined by the direction of the flow. Hence a reversal of the flow direction would imply that the previous outlet becomes the new inlet and vice versa.

If needed, the flow of the liquid can be stopped, and the device will hold the liquid inside the device. Preferably, the microfluidic device comprises a recess forming a holding space in which a substrate surface can be situated. Typically, the volume of this holding space is between 1 and 1000 μL. Preferably the microfluidic device is a device for use in an acoustic force measurement, centrifuge microscopy, flow cytometry or a flow sorting device. More preferably, the microfluidics device is a device for use in an acoustic force measurement and even more preferably the microfluidics device is a device for use in acoustic force spectroscopy (AFS).

A microfluidic device is functionalized on its inside, where the type of functionalization depends on the needs of the specific test to be performed. Depending on the type of device, there may be one or more localized coatings inside the device, meaning that part of the inside is coated and possibly that only part of the inside of the device comprises cellular material, or it may be that the device is fully coated on the inside. In either case, it is preferred to clean the entire inside of the device to ensure that the relevant surfaces, i.e. the measurement or contact surfaces, are clean but also that there are no residual cells left inside the device that might detach and interfere with subsequent measurements. Hence, in a preferred embodiment, the entire inside of the cell is cleaned.

The microfluidic device typically comprises a transparent substrate, e.g., a glass or silica substrate, which may comprise a first coating or primer layer. This primer layer can comprise a physisorbed coating, such as fibronectin or poly-l-lysine (PLL), or a covalent coating, such as a silane. A benefit of using such a primer layer may be improving the binding of further layers to the substrate.

A further biological layer, which is referred to as the target layer, can be applied to the bare or to the primed surface. This layer may comprise viruses, antibodies, peptides, biological tissue factors, antigens, proteins, ligands, or cells, cell organelles, lipid layers, lipid bilayers, cell residues and/or extracellular matrix from cells. Preferably, this layer comprises mammalian cells, mammalian cell organelles, mammalian cell residues and/or extracellular matrix from mammalian cells. This layer may comprise of a single layer of cells (i.e. a monolayer), but may also comprise multiple cell layers (e.g. 3D tissue or organoids). More preferably, this layer consists of mammalian cells, mammalian cell organelles, mammalian cell residues and/or extracellular matrix from mammalian cells. And even more preferably, this layer consists of mammalian cells and/or extracellular matrix from mammalian cells.

Some typical non-limiting examples of mammalian cells comprise tumour cells, stem cells, epithelial cells, B16 melanoma, fibroblasts, endothelial cells, HEK293, HeLa, 3T3, MEFs, HuVECs, microglia, and neuronal cells.

After application of the further biological or target layer, the substrate is normally in a state ready to be incubated. Incubation refers to the process of contacting the target layer with effector cells and allowing the effector cells to bind to the target layer. This can be achieved by flushing a solution of effector cells into the microfluidics device such that the entire holding space is filled with said solution and then, preferably, stopping the flow. This allows the effector cells to bind and/or adhere to the target cells. It is preferred that the effector cells are mammalian cells. Some typical non-limiting examples of effector cells are lymphocytes, monocytic cells, granulocytes, T cells, natural killer cells, B-Cells, CAR-T cells, dendritic cells, Jurkat cells, bacterial cells, red blood cells, macrophages, TCR Tg T-cells, OT-I/OT-II cells, splenocytes, thymocytes, stem cells, BM derived hematopoietic stem cells, TILs, tissue derived macrophages, innate lymphoid cells.

As can be understood, it is preferred that the target cells and/or the effector cells are mammalian cells. In a further preferred embodiment, the target cells and the effector cells are mammalian cells.

In a laboratory setting, it is often unwanted to have living cells in a waste stream as these cells might proliferate and reproduce in an uncontrolled manner. It has been found that ionic liquids are suitable to prevent the proliferation of cells by compromising their membrane integrity. In other words, ionic liquids may cause cell lysis. It is hence preferred that the method according to the invention lyses removed biological matter. More specifically, it is preferred that the removed target cells and effector cells are lysed. Even more specifically, it is preferred that the removed mammalian cells are lysed. It is hereby understood that ionic liquids can lyse both suspended cells and surface-bound cells.

An additional benefit of lysing cells using ionic liquid is that lysed cells are typically easier to remove from the surface compared to living cells. Hence, lysing cells may be beneficial for the cleaning process.

It is understood that, in order for all the biological matter to be detached from the surface, it needs to be in contact with the ionic liquid for a certain period of time. This period is denoted as the contact time. This can be achieved by a continuous flush where ionic liquid continuously flows through the device for the entire contact time at a steady or a varying rate possibly at varying direction, or by an interrupted flush where the flush is started by flowing liquid through the cell, subsequently stopping the flow for a resting period, and optionally restarting the flow of the liquid through the cell.

It is preferred that the contact time is at least 1 minute, preferably at least 2 minutes, preferably at least 5 minutes, preferably at least 10 minutes and, preferably at least 15 minutes, and more preferably at least 20 minutes. It is preferred that the contact time is at most 1 day (1440 minutes), preferably at most 960 minutes, preferably at most 600 minutes, preferably at most 360 minutes, preferably at most 180 minutes, preferably at most 120 minutes, preferably at most 90 minutes, preferably at most 60 minutes, and more preferably at most 30 minutes.

Ionic liquids (ILs) are known in the art and are often defined as compounds chiefly comprising ions with a melting point below a certain temperature. In the context of the current invention, ILs are defined as compounds chiefly comprising ions with a melting point below 100° C. at ambient pressure. Other names for ILs can be liquid electrolytes, ionic melts, ionic fluids, fused salts, liquid salts, or molten salts. ILs are often seen as non-volatile, non-flammable, and air and water stable. ILs have a low vapour pressure, meaning that there is negligible evaporation and no need for the use of ventilated fume hoods.

The cleaning method of the invention uses an ionic liquid to clean biological matter. The method of the invention is preferably an in-situ method wherein the ionic liquid is flushed through the microfluidic device. The ionic liquid hence preferably has a melting point well below room temperature, such as at most −25° C., preferably at most −10° C., or more preferably at least 0° C. The ionic liquid is preferably a thin liquid such that it can easily flow through the device. Preferably, the ionic liquid has a viscosity that allows the ionic liquid to flow through the device using a pressure difference of less than 2 Mpa, more preferably less than 0.5 Mpa, and even more preferably less than 0.1 Mpa.

The method for cleaning a microfluidic device according to the invention is preferably performed at a temperature of between 0 and 100° C., more preferably between 15 and 100° C., even more preferably between 2° and 90° C. and even more preferable between 25 and 75° C. A suitable manner of performing the cleaning at a specific temperature is to control the temperature of the ionic liquid. Hence, in a preferred embodiment of the invention, the temperature of the ionic liquid is between 0 and 100° C., more preferably between 15 and 100° C., even more preferably between 2° and 90° C. and even more preferable between 25 and 75° C.

An ionic liquid is by definition chiefly made up of anions and cations. The ionic liquid used in the method of the current invention preferably comprises at least one cation selected from 1-butyl-1-methyl-pyrrolidinium, 1-ethyl-3-methylimidazolium, and methyltrioctylammonium. More preferably, the ionic liquid used in the method of the current invention preferably comprises 1-ethyl-3-methylimidazolium. The ionic liquid used in the method of the current invention preferably comprises at least one anion selected from chloride, dichloroacetic acid, bromide, iodide, acetate, nitrate, sulfate, sulfonate, trifluoromethansulphonate, tosylate, dicyanamide, thiocyanate, borate, and salicylate. More preferably, the ionic liquids used in the method of the current invention comprises at least one anion selected from dichloroacetic acid, acetate and trifluoromethansulphonate.

The ionic liquids used in the method of the current invention preferably comprise at least one cation selected from 1-butyl-1-methyl-pyrrolidinium, 1-ethyl-3-methylimidazolium, and methyltrioctylammonium and at least one anion selected from chloride, bromide, iodide, acetate, nitrate, sulfate, sulfonate, tosylate, dicyanamide, thiocyanate, borate, and salicylate.

It is hence understood that ionic liquids can suitably be used to remove biological matter inside a microfluidic device. It is also understood that ionic liquids can be used to lyse cells in order to prevent them from proliferating. It is hence of importance to rinse out all ionic liquid from the microfluidic device before performing a subsequent experiment as any residual ionic liquid may influence the results of the subsequent experiment. This rinse can be performed by flushing another liquid through the cell, preferably by flushing a compatible liquid, i.e. a liquid that does not react with the ionic liquid, into which the ionic liquid dissolves. A particularly preferred liquid is an aqueous soap solution. It is hence preferred that the method of the invention further comprises flushing the microfluidic device with an aqueous soap solution. It is understood that flushing with the aqueous soap solution should preferably be performed after flushing with an ionic liquid. In the current context, a soap is used to refer to a surfactant. The term surfactant is known in the art and refers to a molecule having at least a hydrophilic head group and a hydrophobic tail group. Surfactants are often small molecules but, in the context of the current invention larger surfactant molecules such as polymeric surfactants might be preferred. The surfactant can be charged, such as cationic or anionic surfactants, or non-charged, such as non-ionic surfactants. Preferably the soap is a non-ionic surfactant. Particularly preferred non-ionic surfactants are commercially available surfactants under the trade names Pluronics (ex BASF) or Tween (ex Croda).

Additionally, it is preferred that after cleaning with ionic liquid or after rinsing with soap, at least one subsequent rinse with water is performed. The method of the invention hence preferably further comprises flushing the microfluidic device with water, preferably with deionized, demineralized or distilled water.

After the microfluidic device has been cleaned using ionic liquid and preferably subsequently rinsed with an aqueous soap solution and/or water, the device is considered fully cleaned. The surface inside the device is, at least for practical purposes, in such a state that it can be reused for a subsequent measurement. It is hence preferred that the microfluidic device, which is cleaned using the method of the invention, is reused. Preferably, the device can be cleaned at least 10 times, more preferably at least 50 times, even more preferably at least 100 times, and even more preferably at least 150 times without altering the acoustic, hydrodynamical, electrical, optical, and/or surface properties, the inner dimensions and/or its geometry.

Furthermore, it has been found that the cleaning method of the invention does not leave any residue which may alter or negatively affect the (live) cells that are used in subsequent experiments. In other words, the viability and behaviour (for example the measured avidity) of the cells before and after ionic liquid cleaning was the same as far as the inventors could determine.

The present invention hence provides for a method for lysing and removal of biological matter from a surface inside a microfluidic device comprising:
a) flowing an ionic liquid into the holding space of the microfluidic device until the holding space is entirely filled with said ionic liquid,
b) optionally, stopping the flow,
c) optionally, warming the system to a predetermined temperature of 5-100° C. and maintaining said predefined temperature for period of 1-960 minutes,
d) subsequently, removing the ionic liquid from the microfluidic device, preferably by filling the device with air,
e) optionally, rinsing by flowing an aqueous soap solution through the device, and ) optionally, rinsing by flowing through deionized water through the device.

The present invention also provides for a method for lysing and removal of biological matter from a surface inside a microfluidic device comprising:
a) flowing an ionic liquid into the holding space of the microfluidic device until the holding space is entirely filled with said ionic liquid,
b) recycling ionic liquid that has passed at least once through the cell by either reintroducing ionic liquid that has flown trough the outlet into the inlet or reversing the flow direction, c) optionally, warming the system to a predetermined temperature of 5-100° C. and maintaining said predefined temperature for a period of 1-960 minutes,
d) subsequently, removing the ionic liquid from the microfluidic device, preferably by filling the device with air,
e) optionally, rinsing by flowing an aqueous soap solution through the device, and
f) optionally, rinsing by flowing through deionized water through the device.

The above-described method yields a clean device that is in a state ready to be either recoated, reactivated or reused. It is understood that for above method, the biological matter comprises preferably mammalian cells and/or cellular material that is present on the inside of the microfluidic device. The detachment of the biological matter, or the mammalian cells and/or cellular material, takes place at c). The temperature and time at c) are thus carefully chosen to allow for the lysis and detachment of all biological matter.

The invention further encompasses the use of a cleaning agent to remove mammalian cells, mammalian cell residues and/or extracellular matrix from a surface, wherein the cleaning agent comprises an ionic liquid, preferably wherein the cleaning agent consists of an ionic liquid. According to this use, the ionic liquid may comprise at least one cation selected from 1-butyl-1-methyl-pyrrolidinium, 1-ethyl-3-methylimidazolium (EMIM⁺), and methyltrioctylammonium. Also, or additionally, according to this use, the ionic liquid may comprise at least one anion selected from chloride, bromide, iodide, acetate (Ac⁻), nitrate, sulfate, sulfonate (SO₃⁻), trifluoromethanesulfonate (Otf⁻), tosylate, dicyanamide (DCA⁻), thiocyanate, borate, and salicylate.

The invention further also encompasses a kit of parts comprising at least a microfluidic device and a container holding an ionic liquid. In a further preferred embodiment, the kit also comprises a container holding an aqueous soap solution.

As will be evident to the skilled person, different embodiments of the present invention can be combined unless they are mutually exclusive. Many modifications and variations will be apparent to those of ordinary skill in the art without departing from the scope and spirit of the invention The terminology used herein is for the purpose of describing particular embodiments only and is not intended to be limiting of the invention. As used herein, the singular forms “a”, “an”, and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

When amounts, concentrations, dimensions and other parameters are expressed in the form of a range, a preferable range, an upper limit value, a lower limit value or preferable upper and limit values, it should be understood that any ranges obtainable by combining any upper limit or preferable value with any lower limit or preferable value are also specifically disclosed, irrespective of whether the obtained ranges are clearly mentioned in the context.

The examples below will illustrate the practice of the present invention in some of the preferred embodiments. Other embodiments within the scope of the claims will be apparent to one skilled in the art.

### BRIEF DESCRIPTION OF THE DRAWINGS

FIGS. 1A and B representative images of NALM-6 monolayer before and after cleaning with EMIM⁺DCA⁻.

FIGS. 2A and 2B representative images of Skove monolayer before and after cleaning with EMIM⁺Ac⁻.

FIGS. 3A and 3B representative images of Skove monolayer before and after cleaning with EMIM⁺Otf⁻.

FIGS. 4A and 4B representative images of NALM-6 monolayer before and after cleaning with 5% hypochlorite bleach.

FIG. 5 Graphical representation of results of acoustic resonance measurements.

### EXAMPLES

### Experiment 1: Cleaning Target Cells in a Microfluidic Device Using Ionic Liquid

To test the cleaning capability of ionic liquids, an AFS microfluidic device with mimicked use was prepared. The device with mimicked use was prepared by filling a clean device with a cell culture medium comprising target cells. This resulted in the deposition of a cellular monolayer of the target cells.

The microfluidic device with mimicked use was subsequently cleaned. According to the protocol, the culture medium was first removed from the device using negative pressure supplied by a syringe. As a precaution, the monolayer was visually checked using an optical microscope. If a homogeneous layer was observed, the microfluidic device with mimicked use was considered to be a good representation for a used device. The device was subsequently filled with an ionic liquid, heated to the specified temperature, and left for the specified time. Next the device was flushed with an aqueous surfactant solution and subsequently with deionized water. After flushing, the device was visually inspected using an optical microscope to determine the cleanliness of the inner surface.

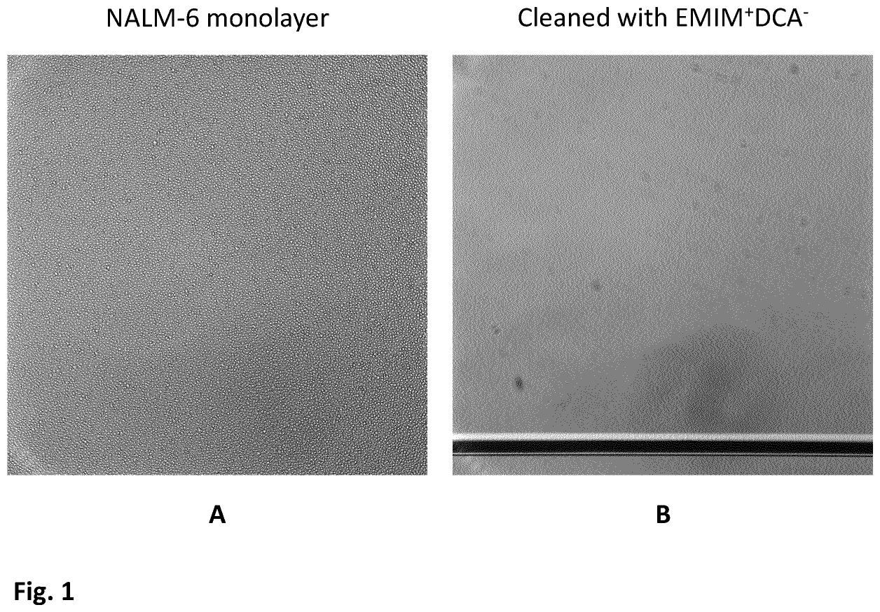

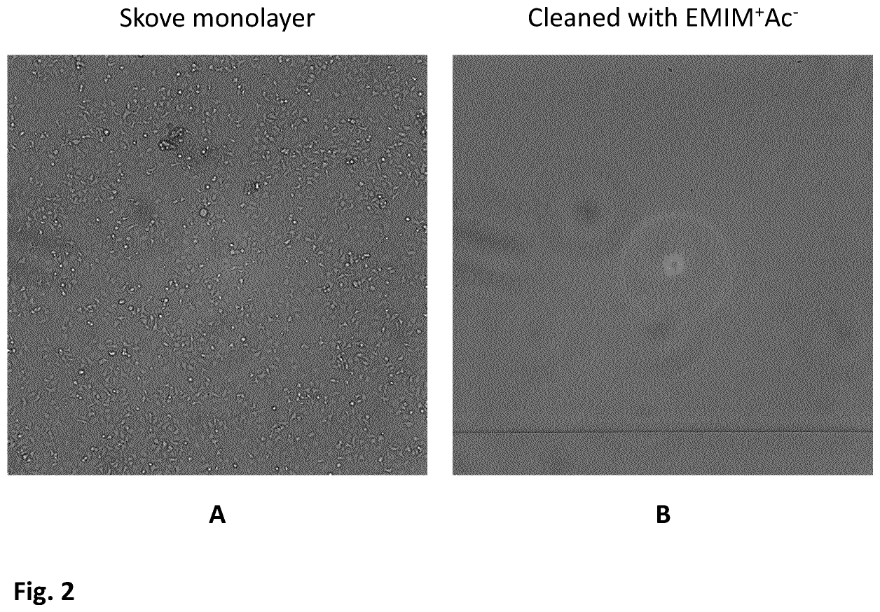

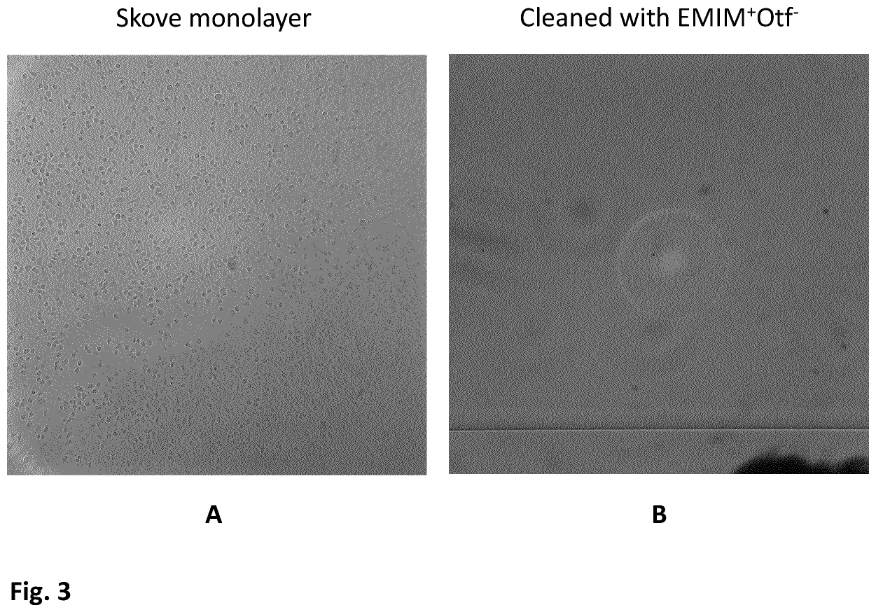

In Table 1, the experimental setup (i.e. the substrate+coating, the target cells, the ionic liquid, the temperature of the experiment, and the holding time) of the experimental program is summarized. In the last column of this table, the result of the cleaning is presented. Representative images of surfaces with mimicked use before and after cleaning are given in FIGS. 1-3.

TABLE 1
Summary of experimental program and result

| Surface | | | Ionic Liquid | | | | |
|---|---|---|---|---|---|---|---|
| Substrate | Coating | Target Cells | Cation | Anion | T (° C.) | t (min) | Result |
| Glass | PLL | NALM-6 | EMIM⁺ | DCA⁻ | 50 | 30 | Clean |
| Glass | PLL | NALM-6 | EMIM⁺ | DCA⁻ | 50 | 300 | Clean |
| Glass | PLL | NALM-6 | EMIM⁺ | DCA⁻ | 15 | 1 | Clean |
| Glass | PLL | NALM-6 | EMIM⁺ | DCA⁻ | 50 | 30 | Clean |
| Glass | PLL | NALM-6 | EMIM⁺ | DCA⁻ | 50 | 210 | Clean |
| Glass | PLL | NALM-6 | EMIM⁺ | DCA⁻ | 50 | 60 | Clean |
| Glass | PLL | Raji | EMIM⁺ | DCA⁻ | 50 | 30 | Clean |
| Glass | PLL | NALM-6 | EMIM⁺ | DCA⁻ | 37 | 30 | Clean |
| Glass | PLL | HeLa | EMIM⁺ | DCA⁻ | 50 | 30 | Clean |
| Glass | PLL | Skove | EMIM⁺ | DCA⁻ | 50 | 30 | Clean |
| Glass | PLL | NALM-6 | EMIM⁺ | DCA⁻ | 50 | 30 | Clean |
| Glass | PLL | Skove | EMIM⁺ | DCA⁻ | 50 | 30 | Clean |
| Glass | PLL | Skove | EMIM⁺ | DCA⁻ | 50 | 60 | Clean |
| Glass | PLL | Skove | EMIM⁺ | Ac⁻ | 50 | 30 | Clean |
| Glass | PLL | Skove | EMIM⁺ | Ac⁻ | 50 | 60 | Clean |
| Glass | PLL | Skove | EMIM⁺ | Otf⁻ | 50 | 30 | Clean |
| Glass | PLL | Skove | EMIM⁺ | Otf⁻ | 50 | 60 | Clean |

### Experiment 2: Cleaning Cells in a Microfluidic Device Using Water or Bleach (Representative)

The above experiment was repeated using water as cleaning medium and using bleach as cleaning medium. It was found that water does lyse at least part of the cells over time (>20 min) but does not solubilize all the released contents. Cleaning with only water hence leaves debris on the glass.

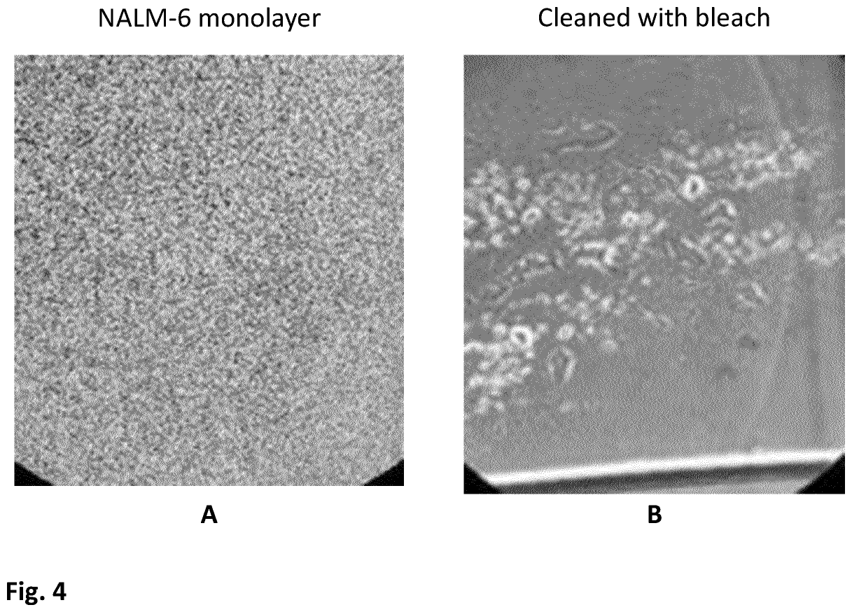

An experiment with bleach was performed using a 5% sodium hypochlorite solution in water at 37° C. for 30 min and a NALM-6 monolayer. It was found that bleach does lyse at least part of the cells over time (>10 min) but does not solubilize all the released contents. Cleaning with only water hence leaves debris on the glass. The visual results of this experiment are shown in FIG. 4.

### Experiment 3: Effect of Cleaning on Acoustic Resonance

The effect of cleaning on the acoustic properties of a microfluidics device was compared when cleaning was performed using a method with harsh chemicals or using ionic liquid (EMIM⁺ DCA⁻).

To determine the effect on acoustic properties when cleaning with ionic liquid, a series of cleaning runs with ionic liquid was performed. The resonance frequency of the device was determined at the beginning (i.e. at n=0 cleaning runs), after 16 cleaning runs and after 110 cleaning runs. To perform a cleaning run, the device was filled with EMIM⁺DCA⁻, heated to 40° C., and left for 60 minutes. Next, the device was flushed with an aqueous surfactant solution and subsequently with deionized water.

To determine the effect on acoustic properties when cleaning with harsh chemicals, a series of cleaning runs with harsh chemicals was performed. The resonance frequency of the device was determined at the beginning (i.e. at n=0 cleaning runs) and throughout the experiment. To perform a cleaning run, the device was filled with bleach and left for 20 minutes at room temperature. Subsequently the device was flushed with water, further rinsed with 12M HCl (required to remove cellular debris), and again flushed with water. Then the device was filled with NaOH 1M and left for 1 h at room temperature. After subsequently flushing with deionized water, the cleaning run was completed.

The resonance frequency of the device was determined by applying a frequency sweep around the expected response frequency and determining the system response. This was performed using the known method such as published in PCT/NL2021/050575 and by Kamsma in section 4.3.1 of his PhD thesis (Kamsma, D 2018, ‘Acoustic Force Spectroscopy (AFS): From single molecules to single cells’, PhD, Vrije Universiteit Amsterdam). Section 4.3.1 is hereby incorporated by reference. According to good practice, the same method and settings for measuring the resonance frequency was used throughout the experiment allowing for a good comparison.

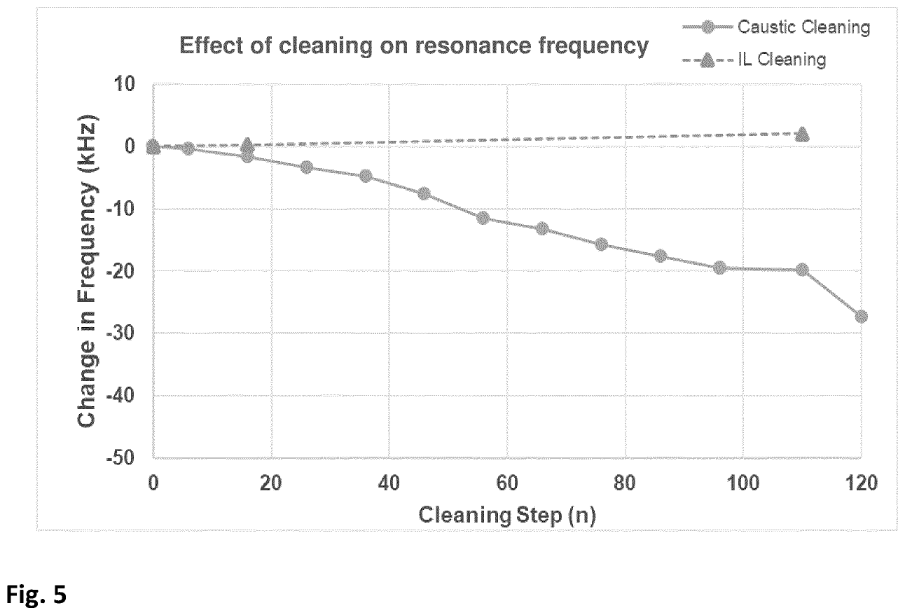

The results of the experiments are summarized in FIG. 5, where the change in resonance frequency (kHz) is shown as a function of the number of cleaning steps (n). As can be seen, the resonance frequency hardly changes when ionic liquid is used for cleaning. However, harsh chemicals do significantly change the resonance frequency.

1. A method for cleaning a microfluidic device comprising removing biological matter from inside of the microfluidic device by flushing the microfluidic device with a cleaning agent, wherein the cleaning agent comprises an ionic liquid.

2. The method according to claim 1, wherein the biological material comprises a component selected from the group consisting of mammalian cells, mammalian cell organelles, mammalian cell residues, extracellular matrix from mammalian cells and combinations thereof.

3. The method according to claim 1, wherein the method for cleaning is an in-situ cleaning method.

4. The method according to claim 1, wherein the ionic liquid comprises at least one cation selected from the group consisting of 1-butyl-1-methyl-pyrrolidinium, 1-ethyl-3-methylimidazolium, and methyltrioctylammonium.

5. The method according to claim 1, wherein the ionic liquid comprises at least one anion selected from the group consisting of chloride, bromide, iodide, acetate, nitrate, sulfate, sulfonate, tosylate, dicyanamide, thiocyanate, borate, and salicylate.

6. The method according to claim 1, wherein a contact time between the microfluidic device and the cleaning agent is at least 5 minutes.

7. The method according to claim 1, wherein the ionic liquid is at a temperature between 15 and 100° C.

8. The method according to claim 1, further comprising a step of reusing the microfluidic device after flushing the microfluidic device with the cleaning agent.

9. The method according to claim 1, wherein the microfluidic device can be cleaned at least 10 times by the method of cleaning without altering the acoustic, hydrodynamic, optical, and/or electrical properties of the device.

10. A method of removing mammalian cells, mammalian cell residues and/or extracellular matrix from a surface, by contacting the surface with a cleaning agent comprising an ionic liquid.

11. The method according to claim 10, wherein the cleaning agent consists of the ionic liquid.

12. The method according to claim 10, wherein the ionic liquid comprises at least one cation selected from 1-butyl-1-methyl-pyrrolidinium, 1-ethyl-3-methylimidazolium, and methyltrioctylammonium.

13. The method according to claim 10, wherein the ionic liquid comprises at least one anion selected from the group consisting of chloride, bromide, iodide, acetate, nitrate, sulfate, sulfonate, tosylate, dicyanamide, thiocyanate, borate, and salicylate.

14. A kit of parts comprising a microfluidic device and a container holding an ionic liquid.

15. The kit of parts according to claim 14, wherein the kit of parts further comprises a container holding an aqueous soap solution.

16. The method according to claim 1, wherein a contact time between the microfluidic device and the cleaning agent is at least 20 minutes.

17. The method according to claim 1, wherein the ionic liquid is at a temperature between 3° and 50° C.

18. The method according to claim 1, wherein the microfluidic device can be cleaned at least 150 times by the method of cleaning without altering the acoustic, hydrodynamic, optical, and/or electrical properties of the device.

19. The method according to claim 1, wherein the biological material comprises a component selected from the group consisting of mammalian cells, mammalian cell organelles, mammalian cell residues, extracellular matrix from mammalian cells and combinations thereof; the ionic liquid comprises at least one cation selected from the group consisting of 1-butyl-1-methyl-pyrrolidinium, 1-ethyl-3-methylimidazolium, and methyltrioctylammonium and the ionic liquid comprises at least one anion selected from the group consisting of chloride, bromide, iodide, acetate, nitrate, sulfate, sulfonate, tosylate, dicyanamide, thiocyanate, borate, and salicylate.

20. The method according to claim 19, wherein the method for cleaning is an in-situ cleaning method.
